(19) United States
(12) Plant Patent Application Publication
(10) Pub. No.: US 2015/0250081 P1
(43) Pub. Date: Sep. 3, 2015

(54) Apricot tree named 'StB14/15'

(50) ;

(71) Applicant: The New Zealand Institute for Plant and Food Research, Auckland (NZ)

(72) Inventors: Arlene E. Nixon, Alexandra (NZ); Michael T. Malone, Havelock North (NZ)

(73) Assignee: The New Zealand Institute for Plant and Food Research, Auckland (NZ)

(21) Appl. No.: 14/544,858

(22) Filed: Feb. 26, 2015

Related U.S. Application Data
(60) Provisional application No. 61/946,596, filed on Feb. 28, 2014.

(51) Int. Cl. A01H 5/00

(52) U.S. Cl. PLT/186

(57) ABSTRACT

A new and distinct apricot variety is described. The variety results from selection among a population of seedlings derived from controlled crossing of the varieties ‘Bhart’ (not patented), marketed as Orangered™ and ‘Late Moorpark’ (not patented). The new variety, StB14/15, is distinguished from others by medium sized fruit with a deep red overcolour when mature accompanied by mid orange coloured firm flesh and low ethylene characteristics. Fruit of StB14/15 matures in early to late February in Otago, New Zealand.

### GENUS AND SPECIES OF PLANT CLAIMED

Prunus armeniaca

### VARIETY DENOMINATION

‘StB14/15’

### CROSS REFERENCE TO RELATED APPLICATION

This application claims benefit of U.S. Provisional Application No. 61/946,596 filed Feb. 28, 2014, the disclosure of which is incorporated by reference in its entirety.

### BACKGROUND OF THE INVENTION

Seedlings obtained from the deliberate crossing of the apricot varieties ‘Bhart’ (not patented), marketed as Orangered™ and ‘Late Moorpark’ (not patented), in September 2001, were planted out at the Clyde Research Station of The New Zealand Institute for Plant and Food Research Limited, where the cross was also conducted. In February 2006, StB14/15 was identified to have potential as a new variety. Later in 2006, StB14/15 was asexually propagated by budding onto ‘Golden Queen’ (not patented) peach seedlings, the standard apricot rootstock in New Zealand. The resulting trees were planted out on the Clyde Research Station in the Southern Hemisphere winter of 2007, and were subsequently found to be true to type demonstrating that the characteristics of the new variety, StB14/15, are stable and transmitted without change through succeeding generations.

### SUMMARY OF THE INVENTION

StB14/15 is characterised by medium sized fruit (70-90 g) with a deep red overcolour, mid orange colour firm flesh and low ethylene characteristics which mature in early-late February in Otago, New Zealand. StB14/15 is distinguished from a number of other varieties by the following characteristics:

StB14/15 has firmer flesh and a longer harvest window when compared to ‘F168’ (U.S. Plant Pat. No. 16,071) (also known as ‘Larclyd’, marketed as Genevieve) grown under the same conditions.

StB14/15 has smaller fruit on average than ‘F194’ (U.S. Plant Pat. No. 16,119), StB 14/15 fruit is approximately 70-90 g while ‘F194’ is approximately 100 g.

StB14/15 can be distinguished from its parent ‘Late Moorpark’ as ‘Late Moorpark’ at harvest maturity has higher soluble solid content, on average 22% while Stb14/15 is 17%. ‘Late Moorpark’ fruit have a tendency to crack at harvest maturity while StB14/15 does not show this tendency.

StB14/15 can be distinguished from its parent ‘Bhart’ as the harvest season of ‘Bhart’ is early while StB14/15 is late. ‘Bhart’ harvests approximately one month earlier than StB14/15.

### BRIEF DESCRIPTION OF PHOTOGRAPHS

The accompanying photographs show typical specimens of the plant, fruit, and leaves of the new variety was depicted in colours as nearly true as is reasonably possible to make the same colour in illustrations of this character.

FIG. 1: Young StB14/15 tree, in summer.

FIG. 2: StB14/15 fruit on the tree.

FIG. 3: StB14/15 whole fruit.

FIG. 4: Longitudinal section of StB14/15 fruit.

FIGS. 5A and 5B: Upper (5A) and underside (5B) of StB14/15 leaves.

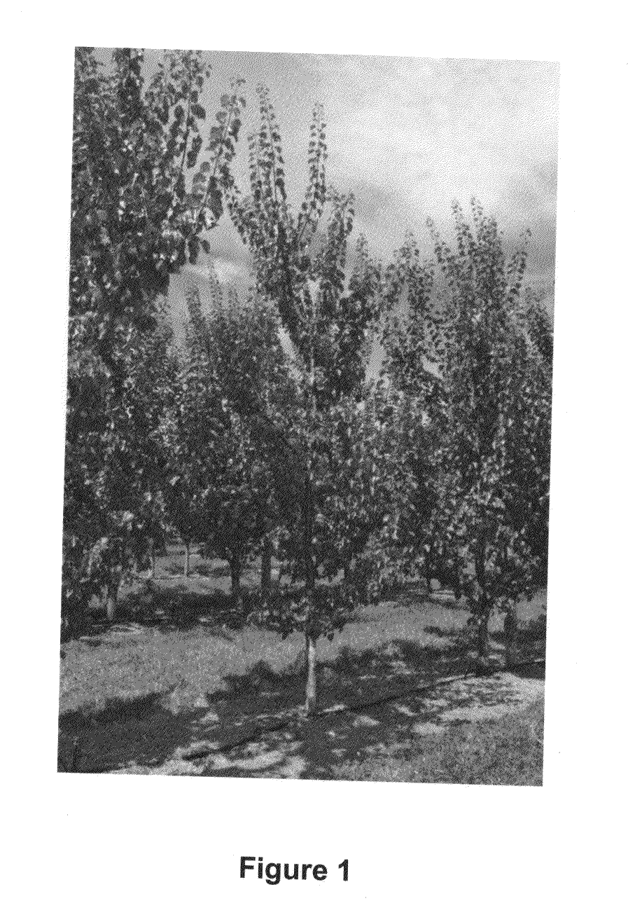

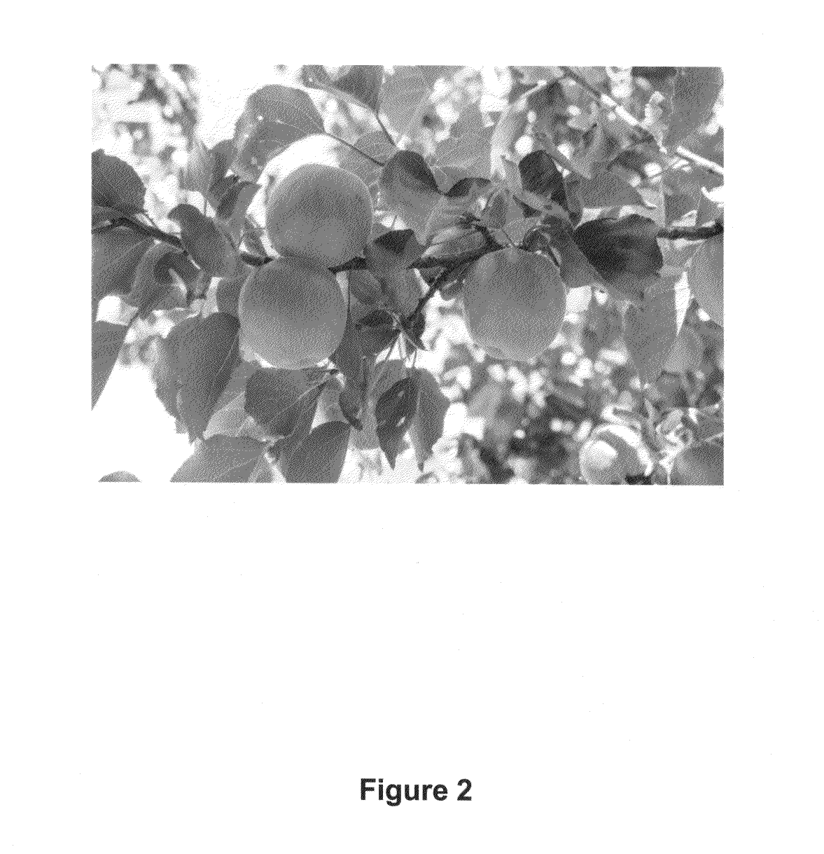

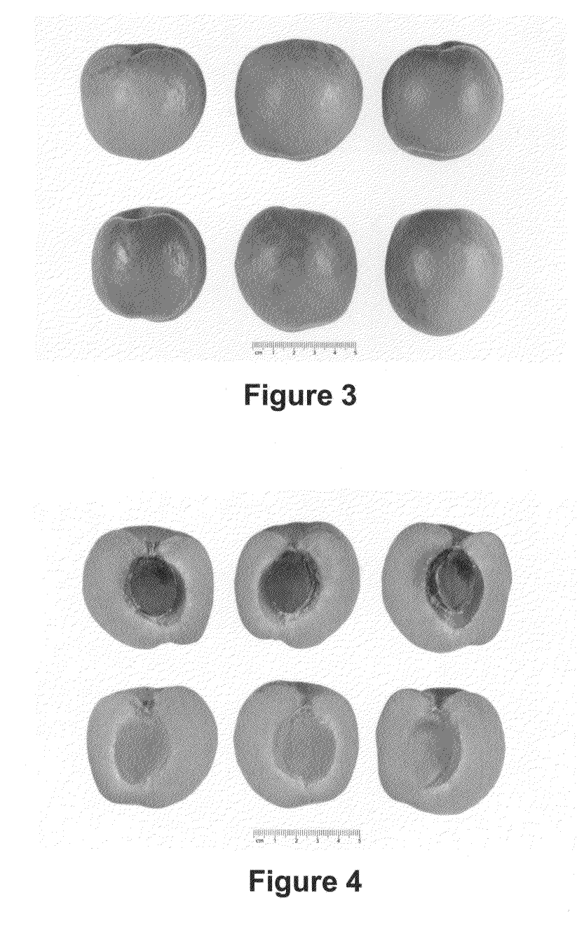

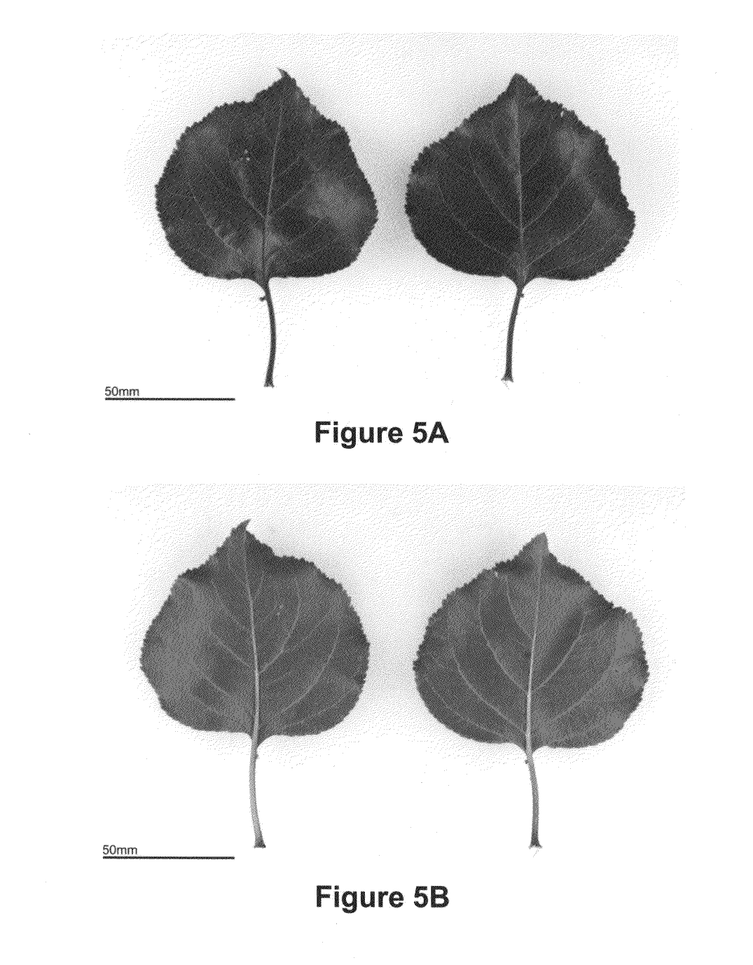

### DETAILED DESCRIPTION

The following is a description of the new variety with colour terminology in accordance with The Royal Horticultural Society Colour Charts (R.H.S.C.C.) Fifth edition (2007). The specimens described were grown in Clyde, New Zealand. The observations were made of the 2013-2015 seasons on 5-7 year old mature trees.

Tree: tree habit is upright to spreading with medium vigour. StB14/15 scions are compatible on peach seedlings (‘Golden Queen’) rootstocks. Tree health is considered to be good under Central Otago conditions in New Zealand with a medium level of precocity compared to commercially grown varieties under similar conditions.

Trunk: The average circumference of a mature trunk, approximately 30 cm from the graft union was 45 cm. The trunk colour was observed to be near brown N200, with a medium rough bark texture, and a medium density distribution of near greyed white 159B lenticular shaped lenticels which averaged approximately 3 mm long×1.2 mm wide.

Leaves: the leaves examined were fully expanded and harvested from the central third of growing shoots in summer. The overall shape of the leaf was observed to be ovate with a broad long acuminate tip approximately 14 mm long, a truncate base and serrate margins with medium undulation. Leaves are arranged alternatively and averaged 85 mm long and 78 mm wide and the profile in cross section was moderately concave. The colour of the upperside was near green 137B with a matt gloss, the colour of the underside was near 147B and the colour of the venation was near yellow green 146D.

Petiole: examined in summer, the length average length of fully expanded leaves was 45 mm and 3 mm in diameter. The upperside of the petioles had anthocyanin colouration of near greyed purple 187B and the underside was near greyed purple 184A. Petioles were observed to have between two and three nectaries close to the leaf base which had a average diameter of 1.2 mm

Flowers: Full bloom, 90% of flowers open, was recorded as occurring in early September (approximately 7 September).

Fruit: fruit at horticultural maturity were medium sized and observed to have an average fruit weight of between 70-90 g with an average height of 55.5 mm, average lateral width of 54.9 mm, and average ventral width of 49.7 mm. StB14/15 fruit were slightly asymmetric and were circular in lateral view and elliptic in ventral view with a slightly sunken suture and a truncate apex. The stalk cavity was medium in depth, approximately 3.2 mm deep. The skin surface was smooth with minimal pubescence, weak gloss, a background colour of near orange 25B and approximately 25-40% overcolour coloured near red 46A made up of a solid flush and isolated flecks. The flesh colour was near orange 25B and near orange 25B in the stone cavity with some whiteness around the stone. The flesh texture was fine and firm, with a weak adherence to the stone and average soluble solids of approximately 17%.

Stone: observations were made on dry stones unless otherwise stated. Stones were ovate in lateral view and had a rounded apex and truncate base. Stones on average weighed approximately 4.2 g, and were approximately 27.2 mm long with an average lateral width of 22.7 mm. The stone was coloured near grey brown 199A.

Harvest timing: fruit begin ripening late in the season, approximately 10-17 February in Clyde, New Zealand.

Use: fresh eating

Pest and disease: StB14/15 has no particular tolerances or susceptibilities to pests and diseases associated with apricot trees.

Keeping quality: has been shown to store very well for at least four weeks at 0° C. StB14/15 is a low ethylene producer, the recorded ethylene production from mature fruit held at 20° C. for 10 days was recorded as approximately 0-0.0008 nmol/kg/s.

1. A new apricot plant substantially as illustrated and described.
